(19) United States
(12) Patent Application Publication
(10) Pub. No.: US 2021/0275363 A1
(43) Pub. Date: Sep. 9, 2021

(54) HEALTH CONDITION SENSOR

(71) Applicant: RSC ASSOCIATES, INC., Bowling Green, OH (US)

(72) Inventors: Thomas Reed STEVENS, Palo Alto, CA (US); Suguru NISHIOKA, I, San Francisco, CA (US); Ivan J. GOERING, Palo Alto, CA (US)

(21) Appl. No.: 17/192,794

(22) Filed: Mar. 4, 2021

Related U.S. Application Data
(60) Provisional application No. 62/984,992, filed on Mar. 4, 2020.

(51) Int. Cl. A61F 13/42; A61B 5/00; A61B 5/20

(52) U.S. Cl. CPC A61F 13/42; A61B 5/0004; A61B 5/0015; A61F 2013/424; A61B 5/20; A61B 2562/02; A61B 5/6804

(57) ABSTRACT

A sensor for detecting the presence of a health condition in a patient, the sensor comprising an absorbent garment or pad, a transmitter including one or more processors, and at least one conductor comprising one or more segments that are separated by absorbent material of the absorbent garment or pad, with the at least one conductor including one or more health condition sensors, where the at least one conductor is configured to detect wetness in the absorbent garment or pad, and where the one or more health condition sensors are configured to detect a urinary health condition in the wetness. Monitoring devices, systems, and methods for detecting a health condition in a patient's urine employing such sensors and health condition sensors are further disclosed.

This application claims the benefit of U.S. Provisional Patent Application No. 62/984,992, filed Mar. 4, 2020, the entirety of which is hereby incorporated by reference.

### BACKGROUND OF THE INVENTION

### Field of the Invention

The present disclosure relates generally to monitoring devices, systems, and methods for detecting a health condition in a patient's urine. More particularly, the present disclosure relates to monitoring devices, systems, and methods for analyzing, tracking, and storing data related to a urinary health condition to promote expedited detection.

### Description of Related Art

Health conditions related to continence, such as urinary tract infections (UTIs), in a patient care environment are a growing and serious problem for elderly patients. Use of special undergarments and absorbent pads or catheterization is the usual practice; however, with limited assistance patients may wait a substantial time in a wet garment or pad for help to change, causing long-term exposure to wetness (e.g., a wet bed, wet underwear, wet clothes, etc.) and significantly increasing the risk of infection. The demand for improved incontinence solutions exist, in ever increasing levels of severity, as the correlation between incontinence and urinary tract infections becomes clear.

However, existing systems have neither been capable of reducing costs nor demonstrated improvement to outcomes associated with incontinence care. For enterprise businesses and caregivers, such as acute care hospitals, incontinence and incontinence management using continence products are a significant expense (e.g., over $4 billion is spent on adult non-woven absorbency products in the US, $9 billion globally), as well as a key source of family dissatisfaction with said care providers. As baby boomers age, there is an increasing and rapidly expanding need to address urinary incontinence and other related health risks (e.g., skin sores, rashes, and infections from skin that is wet or damp, stunted or reduced wound healing, increased susceptibility to fungal infections, etc.). UTIs are a significant and common risk associated with long-term exposure to wet continence products.

### SUMMARY OF THE INVENTION

There currently exists a need for health condition sensor systems for analyzing, tracking, and storing data related to a urinary health condition to promote expedited detection of such health conditions and which can also be easily adapted and integrated to suit individual or multiple patient care facilities. In patient care facilities today, facilities have no way to automatically determine urinary health conditions in a patient, based on the real time and evolving health condition needs of a patient, by automatically detecting a health condition. This leads to increased demands for alternative, health condition sensor based solutions.

Accordingly, there is a need to provide methods and apparatuses for improved incontinence and urinary health condition detection, and a considerable need for sensor systems with improved urinary health condition sensing as well as computing systems that can allow a caregiver to quickly and efficiently address a patient with an acute care need.

Non-limiting embodiments or aspects of the present disclosure are directed to systems, devices, products, apparatuses, and/or methods for a computer-implemented system for analyzing, tracking, and storing data related to a urinary health condition to promote expedited detection. For example, the method may include: detecting, by a health condition sensor, a bacteria in a patient's urine comprising such a health condition by: providing a sensor in a computing system including one or more processors, the sensor including a conductor in an absorbent garment, wherein power is supplied to the conductor to form a circuit; detecting, by the sensor, wetness in the absorbent garment; detecting, by the sensor, a bacteria in the wetness of the absorbent garment; and determining, by the processor, the health condition based on the presence of the bacteria.

According to some non-limiting embodiments or aspects, a health condition sensor detects the presence of a bacteria in a patient's urine, the health condition sensor comprising: an absorbent pad; a transmitter including one or more processors; and at least one conductor comprising one or more segments that are separated by an absorbent material of the absorbent pad. The health condition sensor further comprises the at least one conductor including one or more chemiresistor sensors, wherein the at least one conductor is configured to detect wetness in the absorbent pad, and wherein the at least one conductor is configured to detect the bacteria in the wetness.

The present invention is neither limited to nor defined by the above summary. Rather, reference should be made to the claims for which protection is sought with consideration of equivalents thereto.

Further non-limiting embodiments or aspects will now be described in the following numbered clauses:

Clause 1: A health condition sensor for detecting the presence of a bacteria in a patient's urine comprising: an absorbent pad; a transmitter including one or more processors; and at least one conductor comprising one or more segments that are separated by an absorbent material of the absorbent pad, the at least one conductor including one or more chemiresistor sensors, wherein the at least one conductor is configured to detect wetness in the absorbent pad, and wherein the at least one conductor is configured to detect the bacteria in the wetness.

Clause 2. The health condition sensor according to clause 1, wherein the chemiresistor sensor comprises a carbon allotrope modified with a diazonium compound.

Clause 3: The health condition sensor according to clauses 1 and 2, wherein the carbon allotrope is a carbon nanotube.

Clause 4: The health condition sensor according to clauses 1-3, wherein the carbon nanotube is a single walled carbon nanotube.

Clause 5: The health condition sensor according to clauses 1-4, wherein the at least one conductor comprises a carbon allotrope modified with a diazonium compound.

Clause 6: The health condition sensor according to clauses 1-5, wherein the chemiresistor sensor is configured to detect a health condition based on an electrical impedance in the chemiresistor sensor changing when the at least one conductor is activated with human urine including a bacteria.

Clause 7: The health condition sensor according to clauses 1-6, wherein power is supplied to the at least one conductor to detect wetness in the absorbent garment by: forming a circuit based on wetness bridging between a first segment of the one or more segments and a second segment of the one or more segments, when urine is absorbed in the absorbent pad, wherein the first segment and second segment are connected; or forming a circuit based on wetness bridging between a first segment of the one or more segments and a second segment of the one or more segments, when urine is absorbed in the absorbent pad, wherein the first segment and second segments are individual segments connected in parallel to the transmitter, and wherein absent wetness no circuit exists between the first and second segments.

Clause 8: The health condition sensor according to clauses 1-7, wherein the conductor is configured to change conductivity when activated with a bacteria, wherein a first state is associated with a first impedance and a second state is associated with a second impedance.

Clause 9: The health condition sensor according to clauses 1-8, wherein the conductor comprises a continuous circuit including an initial impedance, wherein wetness including a bacteria on the at least one first conductor changes the impedance to a first state and then to a second state, and wherein the first and second impedance are different from the initial impedance.

Clause 10: A health condition detection method, comprising: providing a sensor in a computing system including one or more processors, the sensor including a conductor in an absorbent garment or pad, wherein power is supplied to the conductor to form a circuit; detecting, by the computing system, wetness in the absorbent garment or pad; detecting, by the computing system, a bacteria in the wetness of the absorbent garment or pad; and determining, by the computing system, a urinary health condition based on the presence of the bacteria.

Clause 11: The health condition detection method according to clause 10, wherein detecting a bacteria further comprises: determining a sensor reading in the sensor based on an activated state of a carbon nanotube.

Clause 12: The health condition detection method according to clauses 10 and 11, comprising: determining that the carbon nanotube is activated by comparing a conductivity reading of the sensor with a previous reading, a stored reading, or a threshold reading of the sensor.

Clause 13: The health condition detection method according to clauses 11 and 12, comprising: determining a sensor reading based on a circuit formed in the conductor and/or the carbon nanotube.

### BRIEF DESCRIPTION OF THE DRAWINGS

FIG. 1 is a diagram of a non-limiting embodiment or aspect of an environment in which systems, devices, products, apparatuses, and/or methods, described herein, can be implemented;

FIG. 2 is a diagram of a non-limiting embodiment or aspect of a system for sensing a health condition;

FIG. 3 is a diagram of a non-limiting embodiment or aspect of components of one or more devices and/or one or more systems of FIGS. 1 and 2;

FIG. 4 is a flowchart of a non-limiting embodiment or aspect of a process for limiting wetness exposure time in an absorbent garment;

FIGS. 5A-5C are diagrams of an implementation of one or more processes disclosed herein;

FIGS. 6A-6C are diagrams of an implementation of one or more processes disclosed herein; and

FIGS. 7A-7C are diagrams of an implementation of one or more processes disclosed herein.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data stored on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices, e.g., an absorbent garment with an associated transmitter and processor, as well as one or more gateways and/or the like, directly or indirectly communicating in the network environment may constitute a “system” or a “computing system.”

It will be apparent that systems and/or methods, as described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Health problems related to incontinence may not be sufficiently addressed by providing tests during doctor's visits. Such infrequent tests may not sufficiently and effectively determine a health condition (e.g., a UTI, etc.) and may not accurately and/or efficiently detect parameters of a health condition (e.g., variations in incontinence, the severity of the health condition, the time of infection, etc.). Relatedly, existing incontinence monitoring systems may have no mechanism for detecting, determining, providing, and/or using health condition information during incontinence events. As well, existing systems may not have or may not accurately and/or efficiently determine information useful for limiting UTI, may not detect a UTI while determining wetness in an absorbent garment, and may not detect wetness in a manner that may efficiently and/or accurately be used to measure, monitor, or store information for determining whether a patient is likely experiencing a UTI. Accordingly, there is a need to provide systems and methods for improved health condition detection, and a considerable need for garments and pads with improved sensing systems that can allow a caregiver to quickly and accurately address a patient with a health condition, to prevent and/or limit a health condition and/or reduce the risk of skin breakdown and infections.

Non-limiting embodiments or aspects of the present disclosure are directed to systems, devices, products, apparatuses, and/or methods for detecting a health condition in an absorbent garment or pad during wetness exposure. For example, the device may include: a health condition sensor for detecting the presence of a bacteria in a patient's urine. The device may further include at least one conductor including one or more segments that are separated by absorbent material, and at least one carbon nanotube (e.g., a conductive ink including a carbon nanotube, etc.). In some non-limiting embodiments or aspects, the conductor may include a carbon nanotube, or alternatively, a carbon nanotube may comprise a bridge between a first contact point and a second contact point of the at least one conductor, wherein the at least one carbon nanotube is activated to conduct electricity when contacted by a bacteria in wetness absorbed in the absorbent garment or pad. In this way, a health condition can be detected and/or electronically diagnosed in a patient care environment more accurately and efficiently, a patient can be automatically diagnosed for improved health remediation, patient garments and pads can more accurately provide a health condition detection for a caregiver to immediately and/or automatically notify or efficiently allow a caregiver to quickly and accurately address a patient with a health condition, and to accurately and/or efficiently determine sufficient information for limiting a health condition by detecting a UTI and/or other health condition while determining a wetness in an absorbent garment or pad in a manner that may efficiently and/or accurately be used to electronically measure, monitor, transmit, or store health information and/or the like.

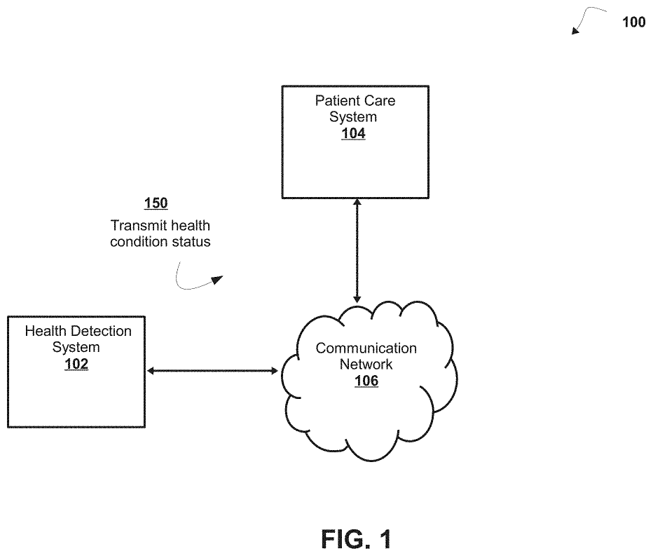

Referring now to FIG. 1, FIG. 1 is a diagram of an example environment 100 in which devices, systems, methods, and/or products, described herein, may be implemented. As shown in FIG. 1, environment 100 includes health condition detection system 102, patient care system 104, and communication network 106. Systems and/or devices of environment 100 can interconnect via wired connections, wireless connections, or a combination of wired and wireless connections.

In some non-limiting embodiments or aspects, health condition detection system 102 includes one or more devices capable of detecting both wetness and the presence of a bacteria in a patient's urine associated with a particular health condition. The health condition detection system 102 includes a transmitter and a wetness sensor having at least one or more conductors, where each conductor may include one or more segments depending on configuration, the segments may be separated by absorbent material, and may include a health condition sensor. The health condition sensor can be configured to provide detection of health conditions associated with wetness absorbed in an absorbent garment or pad that includes, for example, a bacteria. For further example, a health condition sensor can be configured to change electrical conductivity based on contact with another material or substance in urine such as nitrite. The wetness sensor and/or health condition sensor may be disposed within or upon an absorbent material (e.g., a conductor in an absorbent garment, absorbent pad, a multi-layer location-based sensor pad, a multi-layer sensor pad, etc.).

In some non-limiting embodiments or aspects, the health condition detection system 102 includes a wetness sensor, such as, for example, a conductive material formed of a metal and/or a semi-conductive material. In some non-limiting embodiments or aspects, the wetness sensor is configured for detecting wetness only. In some non-limiting embodiments or aspects, a health condition sensor can be included for detecting a health condition. In some non-limiting embodiments or aspects, alternatively and/or additionally, a health condition sensor is integrated into a wetness sensor forming a dual purpose sensor for detecting both wetness and a material or substance present in urine associated with one or more health conditions (e.g., known to correlate with a particular material or substance of a health condition, etc.).

In some non-limiting embodiments or aspects, a transmitter (e.g., a reader, a controller, etc.) connects to the at least one conductor for sending and receiving power to the wetness sensor and/or health condition sensor. The transmitter sending power may provide one or more conductors power for forming a circuit in the sensor to perform the operation of determining whether wetness is present and/or determining whether wetness includes a chemical associated with a health condition. In some non-limiting embodiments, the transmitter includes one or more processors for detecting a change in a reading taken from the conductor. In some non-limiting embodiments, the transmitter may be configured to transmit sensor readings or data to one or more external computing systems (e.g., a gateway, repeater, sensor cloud, patient care system, etc.).

In some non-limiting embodiments or aspects, health condition detection system 102 determines a health condition event based upon a sensor reading, and transmits signals associated with the health condition event via a network 106 (e.g., including one or more of, in any combination, internet services, sensor clouds, hosted or standalone computers, iPads, smartphones, databases, or other transmitters/repeaters/transceivers, etc.). In some non-limiting embodiments, transmitter sends health data associated with the health condition event to a patient care system 104, and/or the like.

In some non-limiting embodiments or aspects, a patient care system 104 receives health condition data from the health condition detection system 102. For example, a patient care system determines a patient status based on health condition data such as an early warning sign of a health condition. In another example, a patient care system 104 may use the health condition data to predict a doctor's prescription before having a definitive lab diagnostic.

In some non-limiting embodiments or aspects, the patient care system 104 includes a user interface for electronic devices (e.g., switches, push buttons, touch screen, Bluetooth transceiver) for receiving information (e.g., data) from a user. For example, patient care system may provide a user interface for one or more electronic devices (e.g., iPads, smartphones, databases, gateways, other transmitters/repeaters/transceivers, etc.) for a user to provide information to a user interface via an electronic device, or for a user to receive information, such as, for example, visual and/or auditory information from the user interface. In some non-limiting embodiments or aspects, patient care system 104 may provide information about the health status of a patient associated with a health condition detection system 102, in addition and/or separately from a detected wetness, a location of a detected wetness, or a status of the health condition detection system 102.

In some non-limiting embodiments or aspects, communication network 106 includes one or more wired and/or wireless networks. For example, communication network 106 includes a cellular network (e.g., a long-term evolution (LTE) network, a third generation (3G) network, a fourth generation (4G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, personal area network (PAN), an ad hoc network, an intranet, the Internet, a fiber optic-based network, a cloud computing network, and/or the like, and/or a combination of these or other types of networks.

The number and arrangement of systems, devices, and networks shown in FIG. 1 are provided as an example. There can be additional systems, devices and/or networks, fewer systems, devices, and/or networks, different systems, devices, and/or networks, or differently arranged systems, devices, and/or networks than those shown in FIG. 1. Furthermore, two or more systems or devices shown in FIG. 1 can be implemented within a single system or a single device, or a single system or a single device shown in FIG. 1 can be implemented as multiple, distributed systems or devices. Additionally, or alternatively, a set of systems or a set of devices (e.g., one or more systems, one or more devices) of environment 100 can perform one or more functions described as being performed by another set of systems or another set of devices of environment 100.

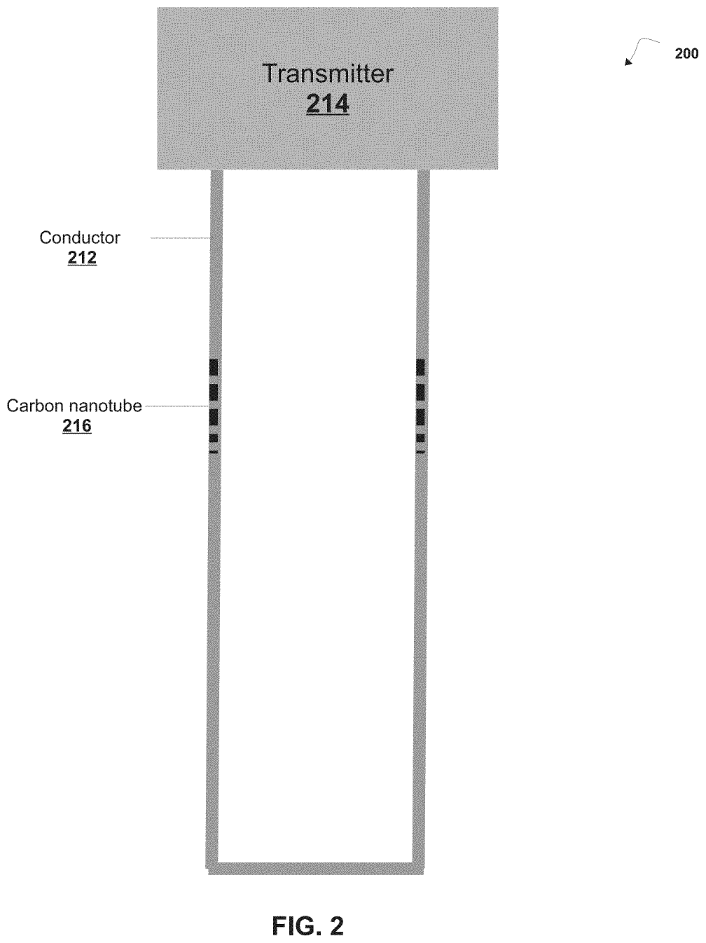

Referring now to FIG. 2, FIG. 2 is a diagram of a non-limiting embodiment or aspect of a sensor 200 for detecting a urinary health condition. As shown in FIG. 2, sensor 200 includes at least one conductor 212, a transmitter 214, and a health condition sensor, such as a carbon nanotube, 216. The conductor 212 may include any conventional structure of a conductor. For example, structures of the conductor, may include a conductor in the form of a conventional wire, a thin-film conductor, a thick-film conductor, and a deposited (e.g. printed, formed by deposits, etc.) conductive material.

In some non-limiting embodiments or aspects, the transmitter 214 may include an interface for receiving a conductor 212 (e.g., an opening, a hinged opening, etc.), the interface configured to include one or more connection pins that are connected with the conductor 212. For example, transmitter 214 forms a circuit by transmitting electricity via a first pin of the one or more connection pins of the interface to the conductor 212. The transmitter 214 receives electricity via a second pin of the one or more connection pins of the interface after it passes through the circuit (e.g., collectively, the conductor 212 and/or health condition sensor 216, etc.) on a pathway (e.g., a path in which electrons from the transmitter flow, etc.) back to the transmitter.

In some non-limiting embodiments or aspects, transmitter 214 determines an initial reading of the circuit after the circuit is formed. For example, transmitter 214 determines an impedance reading of the circuit including the conductor 212 and health condition sensor 216 after an absorbent garment or pad is donned by the patient (e.g., an initial reading, a prior reading, a second reading, etc.).

In some non-limiting embodiments or aspects, the transmitter 214 can detect wetness from incontinence absorbent garments with conductive ink lines. The conductive ink lines can comprise a carbon allotrope, and can themselves include a carbon allotrope (whether the same or a different carbon allotrope) modified with an aromatic compound, such as a carbon nanotube 216.

As used herein, a “carbon allotrope” refers to a material composed of carbon and which has a distinct physical form (i.e., a crystalline structure). Non-limiting examples of carbon allotropes include graphite, amorphous carbon, diamond, fullerenes, carbon nanotubes, aggregated diamond nanorods, glassy carbon, carbon nanofoam, lonsdaleite, and chaoite. In some non-limiting embodiments or aspects, the carbon allotrope is a carbon nanotube such as a single walled carbon nanotube, for example.

In some non-limiting embodiments or aspects, the carbon allotrope is modified with an aromatic compound. As used herein, the term “aromatic” refers to a cyclically conjugated hydrocarbon with a stability (due to delocalization) that is significantly greater than that of a hypothetical localized structure. The carbon allotrope is modified with an aromatic compound by chemically bonding the aromatic compound to the carbon allotrope. For example, the carbon allotrope can include a carbon nanotube, such as a single walled carbon nanotube, and the aromatic compound can be chemically bonded to the outer surface of the carbon wall.

In some non-limiting embodiments or aspects, the aromatic compound can comprise functional groups that will react and bond to the carbon allotrope such as the outer wall of a carbon nanotube. Non-limiting examples of aromatic compounds comprising a functional group and which can be bonded to a carbon allotrope (e.g., the carbon nanotube) include diazonium compounds. The diazonium compound comprises an aromatic structure and a N₂ ion bonded to the aromatic structure which can react with the carbon allotrope (e.g., the carbon nanotube) to bond the aromatic structure thereto.

The aromatic compounds bonded to the carbon allotrope also can comprise additional functional groups that are available for further reactions while they are bonded to the carbon allotrope. These additional functional groups can be selected to react with a desired material or substance including bacteria and other compounds that may be present in urine. Non-limiting examples of additional functional groups include carboxylic acid groups, hydroxyl groups, thiol groups, amine groups, epoxide groups, hydroxyl groups, carbamate groups, amide groups, urea groups, isocyanate groups (including blocked isocyanate groups), and combinations thereof.

In some non-limiting embodiments or aspects, health condition sensor(s) 216 (e.g., a carbon nanotube and/or alternatively, any other carbon allotropes modified with aromatic compounds) can be used as chemiresistors. A “chemiresistor” refers to a material that changes electrical resistance (and, accordingly, conductivity and impedance) in response to changes to its chemical environment. For instance, the carbon allotropes modified with aromatic compounds can interact chemically with substances and materials found in urine to cause a change in electrical resistance, thereby signaling the presence of such materials and substances. Conductive inks including such compounds or structure can be applied in various forms for use in the present invention including, but not limited to, a conductive ink film.

In some non-limiting embodiments or aspects, carbon nanotube 216 chemically activates the conductor (e.g., a conductive film, a conductive ink, a chemiresistor, etc.) to detect the presence of a change in the chemical composition in the nearby environment of a sensor 200. For example, conductor 212 (e.g., a conductive ink, etc.) may include a carbon nanotube 216 to detect materials and substances typically found in urine in connection with a urinary health condition. For example, a bacteria in urine, or a chemical analyte (e.g., a nitrite, etc.), may be used to detect a urinary health condition (e.g., UTI, etc.).

In some non-limiting embodiments or aspects, conductor 212 comprises a sensing material that changes electrical resistance in response to changes in the chemical composition in the nearby environment of the sensor 200 (e.g., metal-oxide semiconductors, conductive polymers, graphene, carbon nanotubes, nanoparticles, etc.). For example, the included carbon nanotube 216 may changes (e.g., deteriorate, degrade, etc.) when a predetermined health condition detected by the sensor 200. For example, by detecting a direct chemical interaction in carbon nanotube 216 between a sensing material and materials or substances present in urine, a urinary health condition may be detected.

In some non-limiting embodiments or aspects, a direct chemical interaction is determined by detecting an interaction of the carbon nanotube 216 with such materials or substances, such as, for example, covalent bonding, hydrogen bonding, molecular recognition, and/or the like.

In some non-limiting embodiments or aspects, the conductor 212 forms an electronic chemical sensor 200. For example, electronic chemical sensor 200 includes carbon nanotube 216 in a space between two electrodes (e.g., two points on a single conductor 212, two points between conductors within a plurality of different conductors, etc.). In some non-limiting embodiments or aspects, conductor 212 comprises carbon nanotube 216 (e.g., one or more carbon nanotubes included in the conductor, etc.). The resistance (or impedance, or conductivity, etc.) between the two electrodes can be measured or monitored to determine various changes in the sensor for determining a status of the absorbent garment or pad.

In some non-limiting embodiments, carbon nanotube 216 includes a sensing material including an inherent resistance that can be modulated by the presence or absence of such materials or substances. During exposure, the materials or substances interact with the sensing material to activate or deactivate the carbon nanotube. These interactions cause change in the resistance reading. In some chemiresistors, an associated resistance changes simply indicate the presence of such materials or substances. In others, the resistance changes are proportional to the amount of such materials or substances present; this allows for the amount of such materials or substances present to be measured.

In some non-limiting embodiments or aspects, transmitter 214 connects to the carbon nanotube 216 via ends of the conductor 212. For example, transmitter 214 transmits and receives electricity on a pathway including both the conductor 212 and the carbon nanotube 216.

In some non-limiting embodiments or aspects, the transmitter 214 determines a reading based on a circuit formed within a plurality of conductors 212. For example, a circuit may be formed in the sensor 200 based on the presence of wetness contacting one or more of a conductor, a conductive film, carbon nanotubes, or chemiresistors formed in the sensor 200, and/or the like.

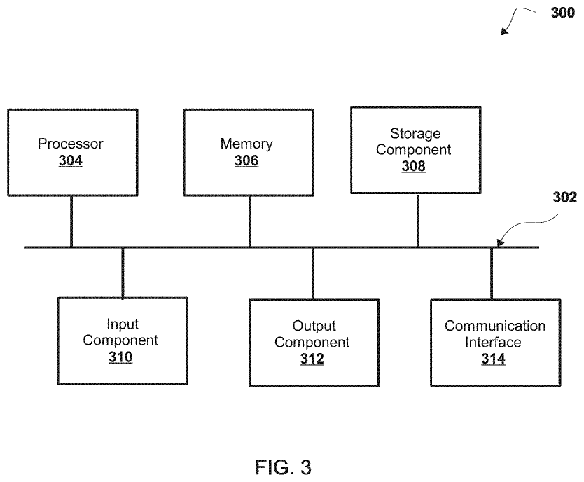

Referring now to FIG. 3, FIG. 3 is a diagram of example components of a device 300 of the present disclosure. Device 300 may correspond to one or more devices of a cloud-implemented method and wetness detection system, one or more devices of the present disclosure that may include at least one device 300, and/or at least one component of device 300. Referring to FIG. 3, the device 300 may include bus 302, processor 304, memory 306, storage component 308, input component 310, output component 312, and communication interface 314. In one embodiment, these elements of a device 300 and the other elements of a device 300 described herein correspond to the cloud-implemented method and wetness detection system, as described herein.

Bus 302 may include a component that permits communication among the components of device 300. In some non-limiting embodiments or aspects, processor 304 is implemented in hardware, firmware, or a combination of hardware and software. For example, processor 304 includes a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 306 may include a random access memory (RAM), a read only memory (ROM), and/or another type of dynamic or static storage device (e.g., flash memory, magnetic memory, optical memory, etc.) that stores information and/or instructions for use by processor 304.

Storage component 308 may store information and/or software related to the operation and use of device 300. For example, storage component 308 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 310 may include a component that permits device 300 to receive information, such as via user input (e.g., a touch screen display, a keyboard, a keypad, a mouse, a button, a switch, a microphone, etc.). Additionally, or alternatively, input component 310 may include a sensor 200 for sensing information. Output component 312 may include a component that provides output information from device 300 (e.g., a display, a speaker, one or more light-emitting diodes (LEDs), an alarm, etc.).

Communication interface 314 may include a transceiver-like component (e.g., a transceiver, a separate receiver and transmitter, etc.) that enables device 300 to communicate with other devices, such as via a wired connection, a wireless connection, or a combination of wired and wireless connections. Communication interface 314 may permit device 300 to receive information from another device and/or provide information to another device. For example, communication interface 314 may include an Ethernet interface, an optical interface, a coaxial interface, an infrared interface, a radio frequency (RF) interface, a universal serial bus (USB) interface, a Wi-Fi interface, a cellular network interface, and/or the like.

Device 300 may perform one or more processes described herein. Device 300 may perform these processes based on processor 304 executing software instructions stored by a computer-readable medium, such as memory 306 and/or storage component 308. A computer-readable medium (e.g., a non-transitory computer-readable medium) is defined herein as a non-transitory memory device. A memory device may include memory space located inside of a single physical storage device or memory space spread across multiple physical storage devices.

Software instructions may be read into memory 306 and/or storage component 308 from another computer-readable medium or from another device via communication interface 314. When executed, software instructions stored in memory 306 and/or storage component 308 may cause processor 304 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software.

The number and arrangement of components shown in FIG. 3 are provided as an example. In some non-limiting embodiments or aspects, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

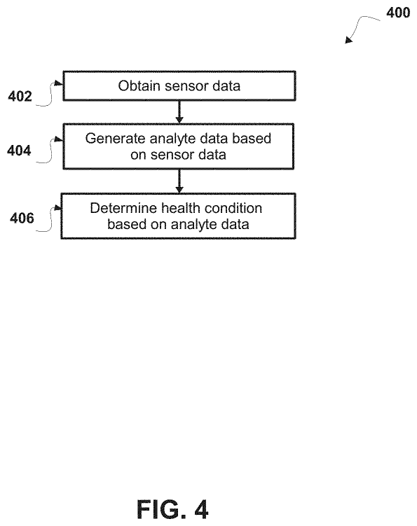

Referring now to FIG. 4, FIG. 4 is a flowchart of a non-limiting embodiment or aspect of a process 400 for determining a urinary health condition of a user of an absorbent garment. In some non-limiting embodiments or aspects, one or more of the steps of process 400 are performed (e.g., completely, partially, etc.) by health condition detection system 102 (e.g., one or more devices of health condition detection system 102, etc.). In some non-limiting embodiments or aspects, one or more of the steps of process 400 are performed (e.g., completely, partially, etc.) by another device or a group of devices separate from or including health condition detection system 102, such as one or more device of (e.g., one or more devices of a system of) patient care system 104.

As shown in FIG. 4, at step 402, process 400 includes obtaining sensor data. For example, health condition detection system 102 obtains sensor data associated with the status of a conductor. In some non-limiting embodiments, health condition detection system 102 determines a sensor reading based on the conductor 212 and the health condition sensor 216, such as the aforementioned carbon nanotube. For example, health condition detection system 102 monitors and/or periodically determines the status based on the power flowing through the conductor 212 and/or the carbon nanotube 216. As an example, the power flowing through the sensor 200 can change based on one or more pathways formed through the conductor 212, the carbon nanotube 216, the absorbent material of the garment, and/or the like.

In some non-limiting embodiments, a sensor can include a continuous circuit or an open circuit. As an example, a continuous circuit includes a conductor configured to receive a continuous power supply (e.g., electrical current, etc.) at one end and capable of conducting the power continuously through the sensor to another end. In such an example, a continuous power supply at one end transfers to a transmitter at another end. In some non-limiting embodiments or aspects, the continuous circuit is associated with an impedance or conductivity of the circuit based on the power passing through to the transmitter. In some non-limiting embodiments, an open circuit includes a plurality of conductors that are not connected. In such an example, a sensor becomes active when an open circuit converts to a closed circuit. For example, an open circuit becomes a closed circuit based on a bridge forming across at least two of the plurality of conductors by wetness received in an absorbent garment.

In some non-limiting embodiments or aspects, a sensor detects wetness in a sensor having an open circuit by detecting the formation of a wetness bridge across two conductors of the sensor. In such an example, the sensor may determine a closed circuit by determining a conductivity or impedance associated with the sensor. The health condition detection system 102 detects wetness based on the sensor converting from an open circuit to a closed circuit. For example, health condition detection system 102 determines wetness based on a jump in conductivity of the sensor associated with wetness bridging at least two conductors.

In some non-limiting embodiments or aspects, health condition detection system 102 determines an initial reading associated with a dry sensor 200 (e.g., a closed circuit, etc.), and the reading includes a baseline of the sensor for when the health condition detection system 102 is in a dry state and has not been activated for a health condition and/or normal wetness. For example, the health condition detection system 102 determines a dry pad state (e.g., when periodically determining a state, etc.) by determining a difference in an impedance since the prior reading.

In some non-limiting embodiments, the transmitter 214 receives and/or determines other wetness events (e.g., a transmitter attachment event, a transmitter detachment event, a sensor wetness event, a sensor dryness event, etc.) while monitoring for a health condition.

In some non-limiting embodiments, health condition detection system 102 is configured to distinguish between normal wetness and health condition wetness associated with a health condition in the wetness. For example, in some non-limiting embodiments, health condition detection system 102 determines normal wetness based on power flowing through the conductor loop 212 and health condition wetness based on the power flowing through the carbon nanotube 216.

In some non-limiting embodiments, health condition detection system 102 determines wetness based on a change in the conductor 212, for example, when wetness is absorbed and one or more pathways are formed as wetness accumulates with the absorbent material adjoining the sensor to create one or more additional pathways. For example, when wetness is absorbed between two conductors forming an open circuit, a wetness bridge may form, where the power can travel through the wetness between two points of the conductor 212. For example, the wetness bridge may create or change the path of the power and thereby change the impedance in the health condition detection system 102.

In some non-limiting embodiments or aspects, health condition detection system 102 senses a change in the system by measuring a change in the health condition detection system 102 based on wetness without materials or substances indicative of a urinary health condition present, and the health condition sensor (e.g., carbon nanotube, other carbon allotrope, etc.) 216 remains inactive until such materials or substances are present in the wetness to thereby activate the health condition sensor 216 and effect a change in the health condition detection system 102 (e.g., the carbon nanotube remains non-conductive until such materials or substances activate the carbon nanotube 216, etc.).

In some non-limiting embodiments and aspects, health condition detection system 102 is configured to sense wetness based on the conductor 212 and the carbon nanotube 216 remains inactive until such materials or substances are present in the wetness to activate the carbon nanotube 216. For example, the carbon nanotube 216 remains non-conductive until materials or substances indicative of a urinary health condition activate the carbon nanotube 216.

In some non-limiting embodiments or aspects, health condition detection system 102 determines wetness based on a change in the conductor 212 and a change in the health condition sensor 216. For example, carbon nanotube 216 is configured to remain conductive until it senses materials or substances indicative of a urinary health condition, thereafter becoming inactive.

In some non-limiting embodiments or aspects, health condition detection system 102 conserves battery power by determining periodically whether there is a detection. For further example, health condition detection system 102 may ignore detections until a jump of conductivity based on the presence of wetness. In such an example, health condition detection system 102 may begin processing impedance detections after wetness is detected to determine the impedance increase or impedance relative to a threshold level for detecting a bacteria.

As shown in FIG. 4, at step 404, process 400 includes generating health condition data based on sensor data formed from readings of the health condition sensor (e.g., carbon nanotube, other carbon allotrope, etc.) 216. For example, health condition detection system 102 is configured to determine a change of conductivity in the carbon nanotube 216 the presence of nitrite in urine that is associated with an increased risk of urinary tract infection.

In some non-limiting embodiments or aspects, the health condition detection system 102 generates health condition data by determining that materials or substances indicative of a urinary health condition are present in the wetness absorbed within an absorbent garment or pad. For example, health condition detection system 102 detects a change in the status of the health condition sensor 216. For example, health condition detection system 102 determines a status change based on a predetermined material or substances, such as a bacteria, a compound, a chemical, a composition, and/or the like that is associated with a urinary health condition by detecting an electro-chemical reaction in a carbon nanotube 216.

In some non-limiting embodiments, health condition detection system 102 may include a time dimension. For example, health condition detection system 102 detects a wetness state of the sensor before determining whether a urinary health condition is detected by the health condition sensor 216.

In some non-limiting embodiments or aspects, health condition detection system 102 is configured to determine the presence of materials or substances, such as, for example, nitrite in urine that is associated with an increased risk of UTI, based on a loss of conductivity in the health condition sensor (e.g., carbon nanotube, other carbon allotrope, etc.) 216. For example, health condition detection system 102 generates health condition data indicating a health condition detection based on a loss of conductivity in the carbon nanotube 216. As an example, health condition detection system 102 is configured to detect less conductivity in the overall system when the carbon nanotube 216 is activated by an analyte, such as nitrite, indicative of an increased risk of UTI.

In some non-limiting embodiments or aspects, health condition detection system 102 determines wetness by comparing two readings, such as comparing one or more prior readings stored in the health condition detection system 102 and an updated reading after a health condition wetness contacts the health condition sensor (e.g., a prior and a current reading from a carbon nanotube, etc.) to determine a difference in the current state of the health condition detection system (e.g., a current or present status, etc.) from a baseline.

In some non-limiting embodiments or aspects, the health condition sensor (e.g., carbon nanotube, other carbon allotrope, etc.) 216 is activated by a health condition wetness. For example, the carbon nanotube 216 activated by the health condition wetness bridging between a first contact point and a second contact point of the conductor 212 based on conductivity formed in the carbon nanotube. In some non-limiting embodiments or aspects, the carbon nanotube 216 activated by the health condition wetness eliminates an existing conductivity bridge between a first contact point and a second contact point of the conductor 212. In some examples, activation of the carbon nanotube 216 changes an already existing status (i.e., lowers or increases conductivity or impedance through the carbon nanotube, etc.)

In some non-limiting embodiments, health condition detection system 102 determines an impedance after a health condition sensor (e.g., carbon nanotube, other carbon allotrope, etc.) has been activated. As an example, health condition detection system 102 determines a resistance based on a degradation (e.g., decay, etc.) in the structure of a conductor (e.g., conductive ink, chemiresistor, etc.). In some examples, a carbon nanotube activated with health condition wetness begins to break down a conductor. In some non-limiting embodiments or aspects, health condition detection system 102 determines a health condition by measuring a sensor including a degradation in the conductor (e.g., a conductive ink, etc.) based on a carbon nanotube destroying or degrading the structure of the ink, such that, for example, a conductivity decreases or stops.

In some non-limiting embodiments or aspects, health condition detection system 102 includes a health condition sensor (e.g., carbon nanotube, other carbon allotrope, etc.) 216 that reacts to health condition wetness over a period of time. For example, a period of time can be measured or sensed based on a time between initial wetness detection and a period at which and/or during when a health condition sensor reading is changing because of interactions with a bacteria.

As shown in FIG. 4, at step 406, process 400 includes determining a health condition based on health condition data. For example, health condition detection system 102 determines a urinary health condition based on health condition data associated with a UTI. As an example, health condition detection system 102 determines urinary health condition data based on a detection of materials or substances that are associated with a urinary health condition and provides the health condition data to a patient care facility 104.

In some non-limiting embodiments or aspects, transmitter 214 sends a reading associated with the status of the sensor within an absorbent garment or pad to the gateway/cloud by determining a range of the reading. For example, transmitter 214 determines a reading of the status by detecting a change in impedance.

In some non-limiting embodiments, transmitter 214 includes a gateway, or alternatively, connects to a gateway and/or cloud system for sending health condition data associated with a health condition event (e.g., sending health condition event to a sensor cloud, a patient care system, etc.). For example, in some non-limiting embodiments, a local gateway receives health condition data. The gateway provides an interface to store and/or forward health condition data to a patient care facility. In another example, the gateway supplements the health condition data with health condition detection system data, such as, for example, IP addresses, timestamps, gateway identifiers, network identifiers, transmitter identifiers, and/or the like.

In some non-limiting embodiments or aspects, health condition detection system 102 is configured to detect one or more conditions in the absorbent garment or pad (e.g., no wetness, wetness, wetness with elevated pH, wetness with elevated level of nitrite, wetness with an analyte indicative of UTI, etc.). In further examples, health condition detection system 102 detects a health condition, such as dehydration (e.g., wetness with a depressed pH associated with dehydration, etc.) and/or UTI. As an example, health condition detection system 102 may include a health condition sensor that is configured to detect a pH associated with dehydration based on a delta impedance after initial wetness is detected (e.g., a first delta that is less than the delta for UTI, etc.). In such an example, health condition detection system 102 determines a UTI based on an increase in impedance (e.g., a second delta associated with a UTI, etc.) In some examples, health condition detection system 102 also determines urinary health condition, including UTI and/or dehydration, based on a timing of a change in impedance after an initial wetness is determined.

In some non-limiting embodiments or aspects, health condition detection system 102 includes a sensor having an open circuit. In such an example, the at least one conductor is configured as an open circuit. In some non-limiting embodiments or aspects, after a circuit is closed, there is an impedance detection because power can move to the opposite side of the circuit (i.e., another side of the pin connection of the transmitter to the circuit). For example, the open circuit closes based on wetness (e.g., an initial wetness detection from a patient's urination absorbed into an absorbent pad including a sensor, etc.). In some non-limiting embodiments or aspects, health condition detection system 102 determines a detection in coordination with a timing of the initial wetness detection. For example, health condition detection system 102 is programmed or configured with a predetermined interval for timing a change from wetness to a health condition reading based on a time for the health condition sensor to react to bacteria. In such an example, health detection system includes a transmitter for detecting an impedance to start a timer. In some non-limiting embodiments or aspects, health condition detection system 102 determines a range of impedance readings. In some non-limiting embodiments, health condition detection system 102 determines a closed circuit based on an initial impedance reading (e.g., in an open circuit sensor, etc.). For example, a sensor having an open circuit is associated with a large impedance change between an initial impedance (e.g., an impedance associated with complete resistance because a circuit is open, etc.) and a wetness impedance that is associated bridging at least two conductors of an open circuit. In such an example, health condition detection system 102 includes a transmitter that is configured to determine that a bridge has formed between two conductors of the open circuit such that the open circuit has become closed.

In some non-limiting embodiments or aspects, health condition detection system 102 transmits a category based on determining that a circuit has become closed in the sensor. In such an example, health condition detection system 102 monitors (e.g., periodically monitors, etc.) an open circuit until it becomes closed, and then transmitter begins to transmit the category.

In some non-limiting embodiments, health condition detection system 102 includes a closed circuit where impedance decreases upon exposure to wetness. For example, health condition detection system 102 includes a transmitter that is configured to determine whether a section of a conductor (e.g., a segment, a part, etc.) has been closed.

In some non-limiting embodiments or aspects, health condition detection system 102 determines a health condition delta (e.g., a threshold number, a range of continuous detections from a time of activation of the carbon nanotube, etc.). For example, a delta may be determined based on how a conductor (e.g., a conductive ink, a conductive film, a chemiresistor, a carbon nanotube, etc.) reacts when activated with wetness. Health condition detection system 102 may determine a threshold number and/or store a threshold number. Health condition detection system 102 may determine a range of continuous detections based on a plurality of measurements taken between an initial wetness is detected and when a higher impedance is detected.

In some non-limiting embodiments or aspects, health condition detection system 102 includes detection of multiple categories. In some examples, health condition detection system 102 determines an impedance range associated with a condition category. For example, a diagnostic category such as likelihood, progression, or severity is associated with a one or more ranges of impedance. In such an example, the health condition detection system 102 may determine an increase in impedance (in particular, a change in category) in comparison to prior readings of impedance.

In some non-limiting embodiments, health condition detection system 102 includes a transmitter configured to send categories. For example, a transmitter may send messages to a sensor cloud and/or gateway for processing periodically, such as, “this is category 1,” “this is category 4,” and “this is category 5.” In such an example, a cloud or gateway may receive and process the messages to determine a judgement (e.g., determine a health condition based on a delta, etc.).

In some non-limiting embodiments, health condition detection system 102 includes a transmitter that matches the impedance to a range of a category. As an example, a wetness measurement may change between category 1, 2, 3, but if the wetness measurement is outside of categories 1, 2, 3 it is not normal wetness. In some non-limiting embodiments or aspects, health condition detection system 102 may include a sensor having a delay or not having a delay. For example, health condition detection system 102 may include a sensor having a delay, and based on a first detection of wetness, health condition detection system 102 determines a second detection associated with the delay. In some examples, the delay is caused by a sensor element such as a carbon nanotube forming an increased resistance over a period of time. The resistance may be undetectable until a sufficient time period is met, such as, the time it takes for a conductor to degrade.

In some non-limiting embodiments or aspects, health condition detection system 102 may not include a delay. As an example, health condition detection system 102 may be programmed or configured to determine a health condition based on a delta. For example, health condition detection system 102 may determine that a wetness measurement (e.g., impedance, etc.) has jumped between categories 1, 2, and 3. In such an example, health condition detection system 102 determines if the delta is within the same range. In some examples, health condition detection system 102 determines a category of 5 to indicate a pH, a category of 7 (e.g., 7 or higher, etc.) to indicate a UTI. For example, a delta of 3 categories may be used to determine that if category 1 is the dry state, category 4 is declared wet, category 7 is pH, and/or category 10 and up is UTI. In some non-limiting embodiments, health condition detection system 102 determines a category and sends it to a sensor cloud for judging the wetness.

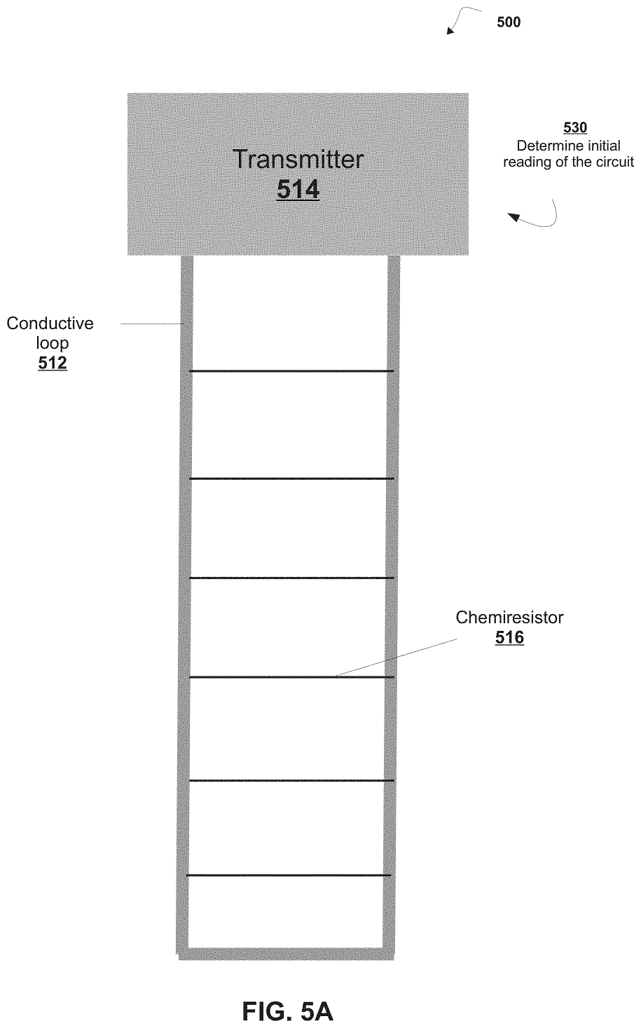

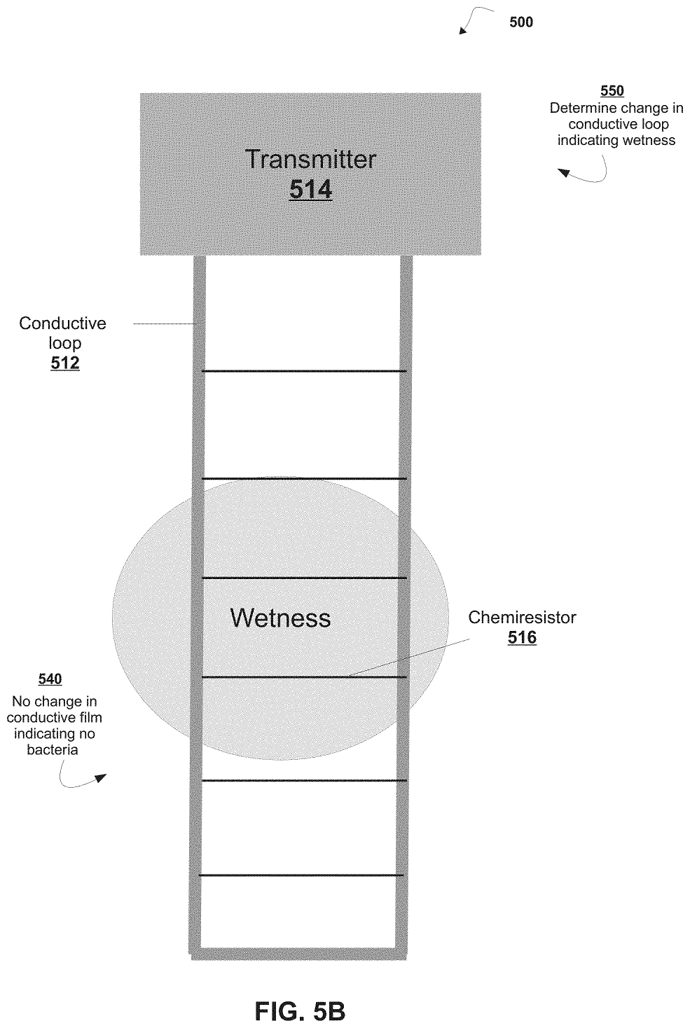

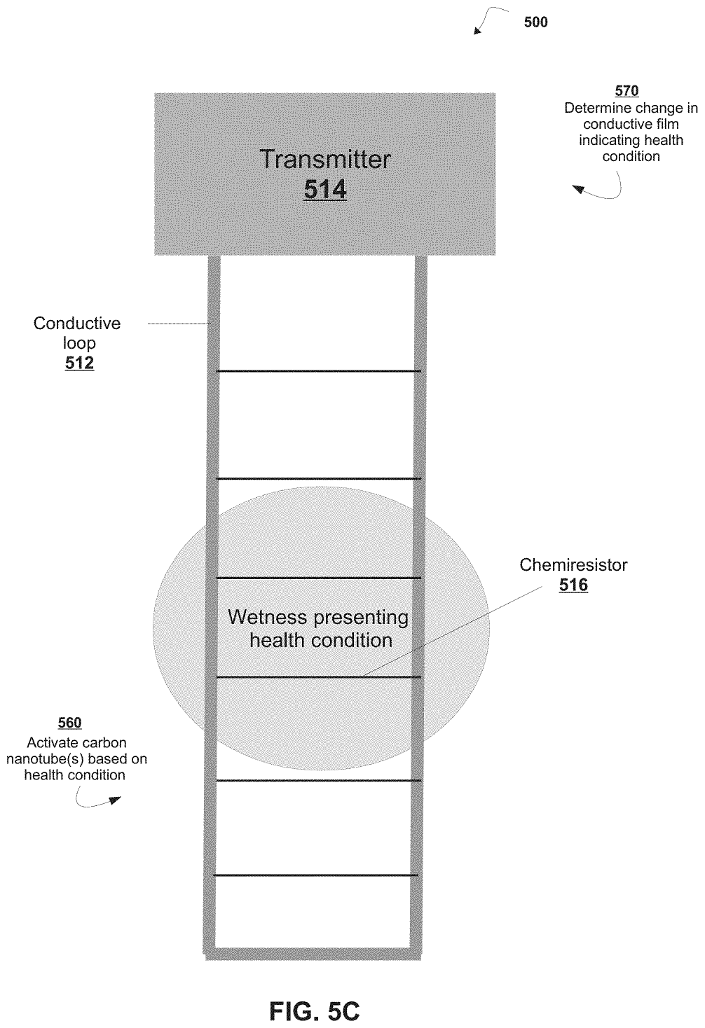

Referring now to FIGS. 5A-5C, FIGS. 5A-5C are diagrams of an overview of a non-limiting embodiment of a sensor 500 relating to one or more processes disclosed herein. Implementations using the sensor 500 may include a health condition detection system and patient care facility. In some non-limiting embodiments or aspects, health condition detection system can be the same as or similar to health condition detection system 102. In some non-limiting embodiments or aspects, patient care facility can be the same as or similar to patient care facility 104. In some non-limiting embodiments or aspects, conductor 512 can be the same as or similar to conductor 212. In some non-limiting embodiments or aspects, health condition sensor 516 can be the same as or similar to health condition sensor 216. In some non-limiting embodiments or aspects, transmitter 514 can be the same as or similar to transmitter 214.

As shown by reference number 530 in FIG. 5A, sensor 500 determines an initial reading of a circuit formed by conductor 512. For example, when an absorbent garment or pad including the sensor 500 is donned by the patient, an initial reading is measured by the transmitter 514. The initial reading may be stored in the health condition detection system, such as transmitter 514, a gateway, or some other processing device.

As shown by reference number 540 in FIG. 5B, when wetness is detected in the absence of a predetermined health condition detectable by a health condition sensor 516, sensor 500 indicates no health condition sensor change is detected (e.g., in a carbon nanotube 516 sensitive to the presence of bacteria, no change indicating the presence of a bacteria). Wetness that is present is detected by the conductor 512 and forms a pathway through the conductor 512, absorbed wetness, and/or the like. As wetness is present, the sensor changes state, but illustrated carbon nanotube 516 is not activated as bacteria (or materials or substances indicative of a urinary health condition, as described earlier above) are not present in the wetness, therefore a health condition is not determined.

As shown by reference number 550 in FIG. 5B, sensor 500 determines a change in conductor 512 indicating wetness proximate the sensor. For example, a reading of the sensor 500 determines that wetness is present in the environment of the conductor 512. However, the sensor 500 does not detect a health condition.

As shown by reference number 560 in FIG. 5C, wetness may alternately present a condition that activates the health condition sensor 516 based on a health condition. For example, a wetness including a predetermined material or substance, such as nitrite for the detection of UTI, may be determined. Wetness is detected by the conductor 512 and forms a pathway through the conductor 512, absorbed wetness, and/or the like. As wetness is present, the sensor 500 changes state, but also as indicated by 570, a health condition is determined.

As shown by reference number 570 in FIG. 5C, the sensor 500 determines change in one or more health condition sensors 516 indicating that a health condition is present. As a result of a change in health condition sensor(s) 516, the pathway of power through the conductor 512 may change (e.g., conduction through a carbon nanotube, other carbon allotrope, chemiresistor 516, etc. may create a “short” across the conductor 512 detectable as a change in conductance, resistance, or impedance) or the character of the pathway may change (e.g., conduction through a carbon nanotube, other carbon allotrope, chemiresistor 516, etc. may increase relative to conduction through wetness alone, detectable as a change in conductance, resistance, or impedance). The change in one or more health condition sensors 516 may be indicative of a particular category depending upon the location of the health condition sensor and, thus, the change in the pathway of power through the circuit.

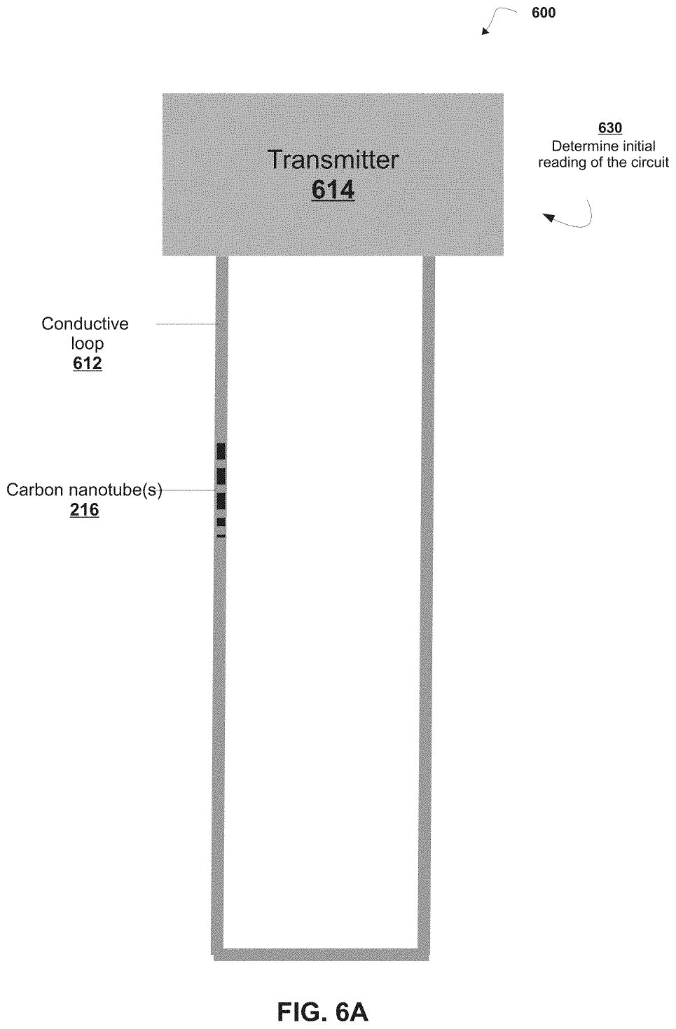

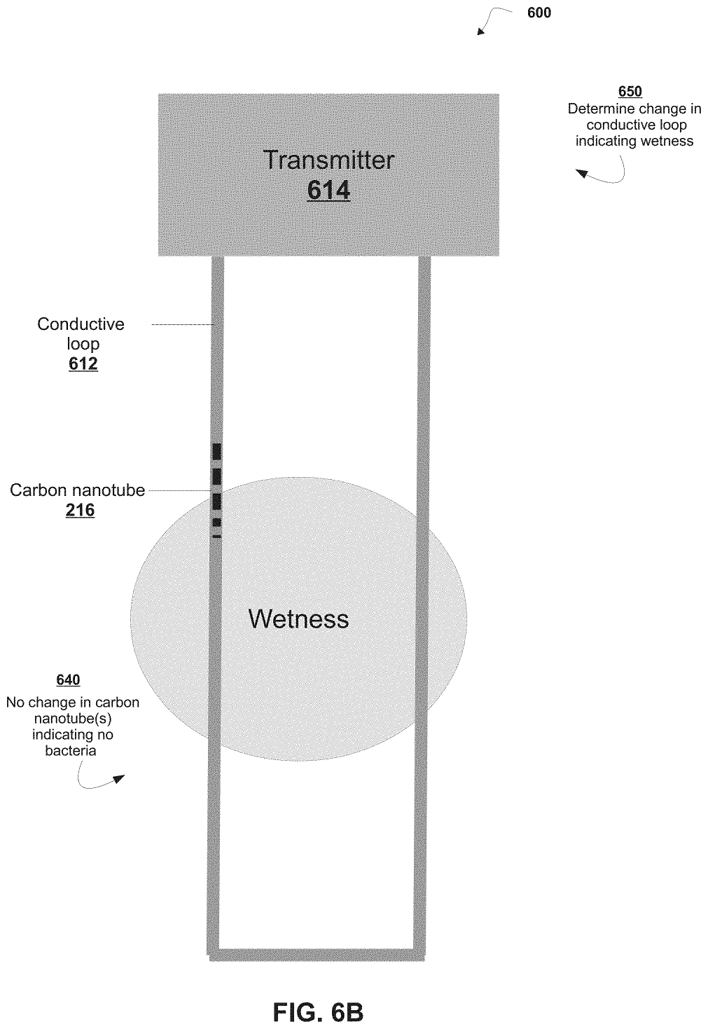

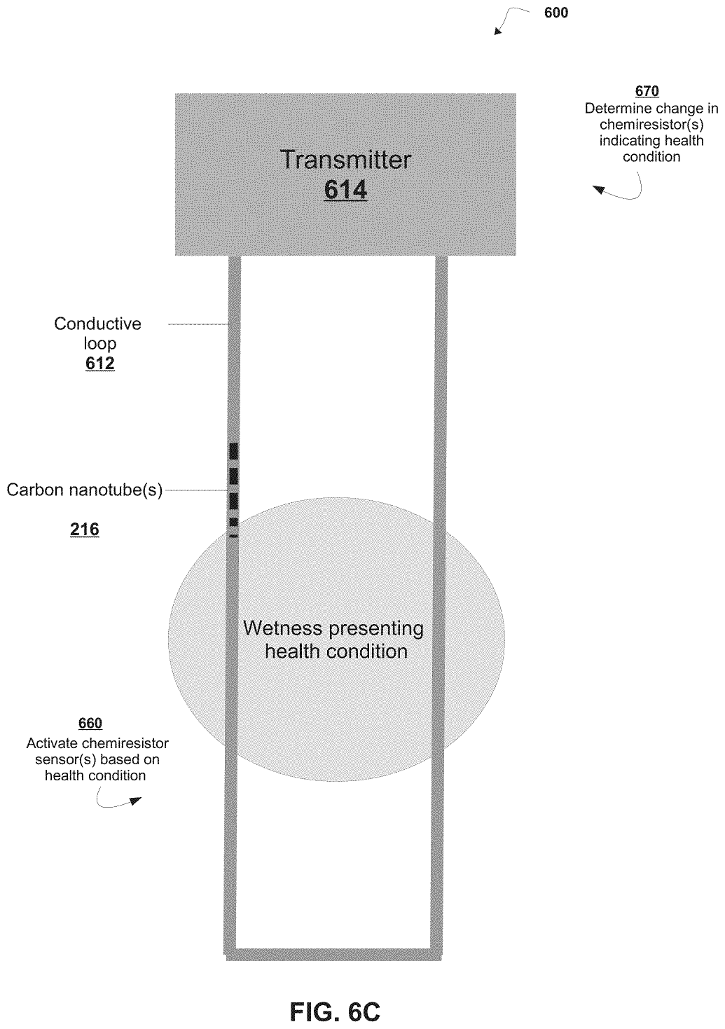

Referring now to FIGS. 6A-6C, FIGS. 6A-6C are diagrams of an overview of a non-limiting embodiment of a sensor 600 relating to one or more processes disclosed herein. Implementations using the sensor 600 may include a health condition detection system and a patient care facility. In some non-limiting embodiments or aspects, health condition detection system can be the same as or similar to health condition detection system 102. In some non-limiting embodiments or aspects, patient care facility can be the same as or similar to patient care facility 104. In some non-limiting embodiments or aspects, conductor 612 can be the same as or similar to conductor 212. In some non-limiting embodiments or aspects, health condition sensor 616 can be the same as or similar to health condition 216. In some non-limiting embodiments or aspects, transmitter 614 can be the same or similar to transmitter 214.

As shown by reference number 630 in FIG. 6A, sensor 600 determines an initial reading of a circuit formed by a conductor 612. For example, when an absorbent garment or pad including the sensor 600 is donned by the patient, an initial reading is measured by the transmitter 614. The initial reading may be stored in the health condition detection system, such as transmitter 614, a gateway, or some other processing device.

As shown by reference number 640 in FIG. 6B, when wetness is detected in the absence of a predetermined health condition detectable by a health condition sensor 616, sensor 600 indicates no health condition sensor change is detected (e.g., in a carbon nanotube 616 sensitive to the presence of bacteria, no change indicating the presence of a bacteria in the absorbed wetness). Wetness that is present is detected by the conductor 612 and forms a pathway through the conductor 612, the health condition sensor 616, the wetness, or a combination. As wetness is present, the sensor changes state, but illustrated carbon nanotube 616 is not activated as bacteria (or materials or substances indicative of a urinary health condition, as described earlier above) are not present in the wetness, therefore a health condition is not determined.

As shown by reference number 650 in FIG. 6B, sensor 600 determines a change in conductor 612 indicating wetness proximate the sensor. For example, a reading of the sensor 600 determines that wetness is present in the environment of the conductor 612. However, the sensor 600 does not detect a health condition.

As shown by reference number 660 in FIG. 6C, wetness may alternately be present in a condition that activates the health condition sensor 616 based on a health condition. For example, a wetness including a predetermined material or substance, such as nitrite for the detection of UTI, may be is determined. As wetness is present, the sensor 600 changes state, but also as indicated by 670, a health condition is determined.

As shown by reference number 670 in FIG. 6C, the sensor 600 determines a change in the health condition sensor 616 indicating that a health condition is present. Change in health condition sensor 616 may change the character of conduction through the conductor 612 (e.g., conduction through a carbon nanotube, other carbon allotrope, chemiresistor 616, etc. may increase or decrease relative to conduction through wetness alone, detectable as a change in conductance, resistance, or impedance).

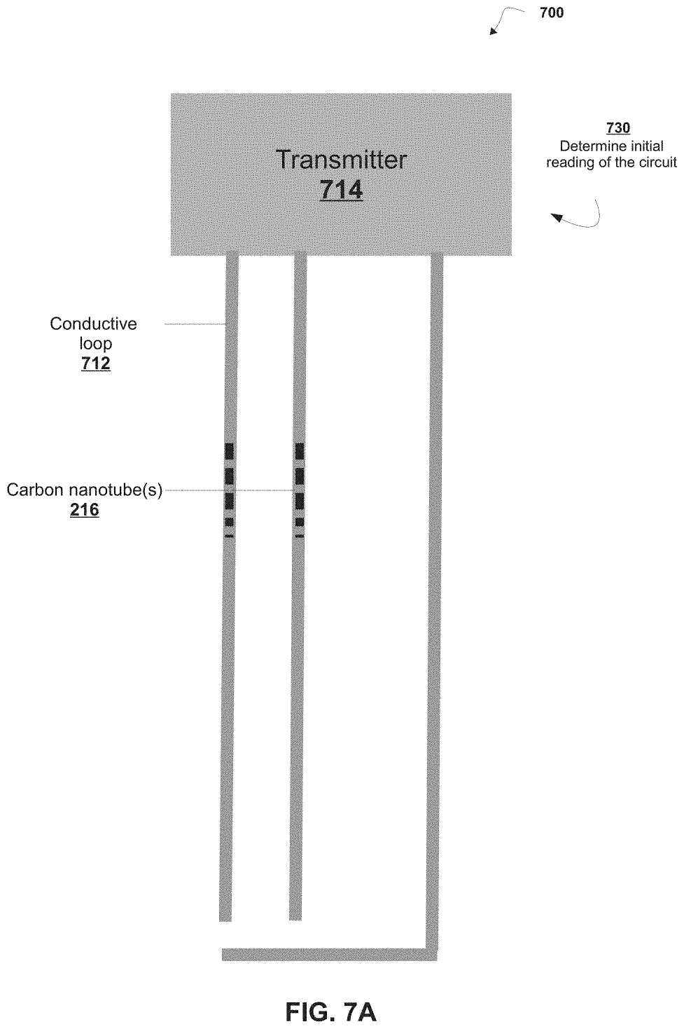

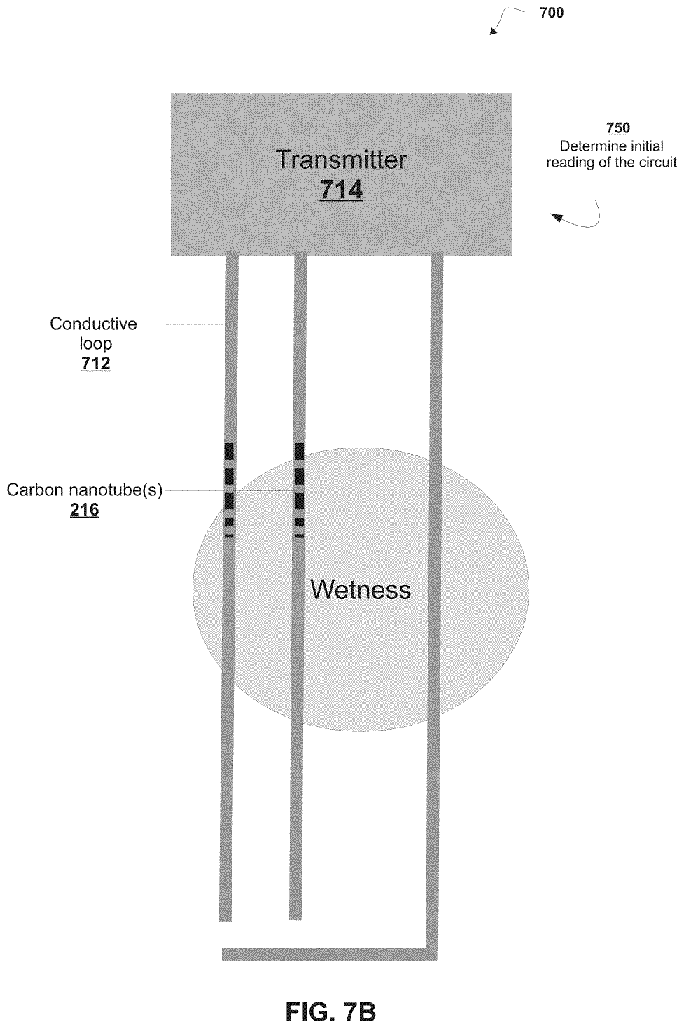

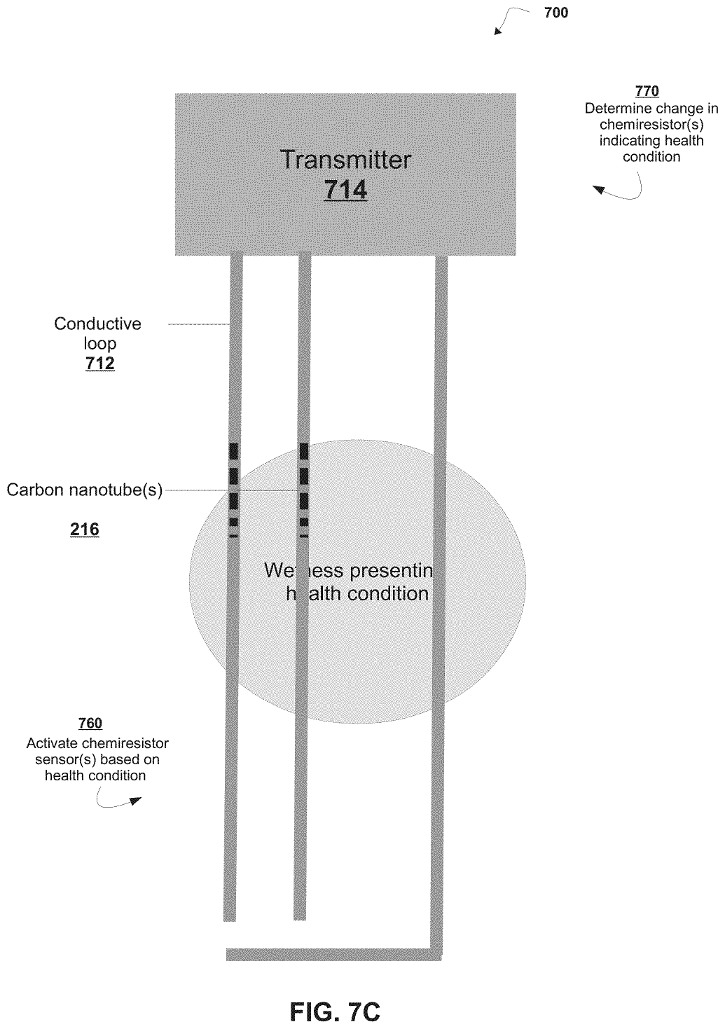

Referring now to FIGS. 7A-7C, FIGS. 7A-7C are diagrams of an overview of a non-limiting embodiment of a sensor 700 relating to one or more processes disclosed herein. Implementations using the sensor 700 may include a health condition detection system and a patient care facility. In some non-limiting embodiments or aspects, health condition detection system can be the same as or similar to health condition detection system 102. In some non-limiting embodiments or aspects, patient care facility can be the same as or similar to patient care facility 104. In some non-limiting embodiments or aspects, conductor 712 can be the same as or similar to conductor 212. In some non-limiting embodiments or aspects, health condition sensor 716 can be the same as or similar to health condition sensor 216. In some non-limiting embodiments or aspects, transmitter 714 can be the same as or similar to transmitter 214.

As shown by reference number 730 in FIG. 7A, sensor 700 determines an initial reading of circuits formed by a plurality of conductors 712. For example, when an absorbent garment or pad is donned by the patient, initial readings are measured by the transmitter 714 (e.g., between left conductor 712 and right conductor 712, and middle conductor 712 and right conductor 712). The initial readings may be stored in the health condition detection system, such as transmitter 714, a gateway, or some other processing device.

As shown by reference number 740 in FIG. 7B, when wetness is detected in the absence of a predetermined health condition detectable by a health condition sensor 716, sensor 700 indicates no health condition sensor change is detected (e.g., in a carbon nanotube 716 sensitive to the presence of bacteria, no change indicating the presence of a bacteria in the absorbed wetness). Wetness that is present is detected by the conductor 712 and forms a pathway through the conductor 712, the health condition sensor 616, the wetness, or a combination, and the impedance amongst the plurality of conductors changes. As wetness is present, the sensor changes state, but illustrated carbon nanotubes 716 (whether within the left or middle conductors 712) are not activated as bacteria (or materials or substances indicative of a urinary health condition, as described earlier above) are not present in the wetness, therefore a health condition is not determined.

As shown by reference number 750 in FIG. 7B, sensor 700 determines a change in the plurality of conductors 712 indicating wetness proximate the sensor. For example, a reading of the sensor 700 determines that wetness is present in the environment of the plurality of conductors 712. The reading may be between the left conductor 712 and the right conductor 712, the middle conductor 712 and the right conductor 712, a combination of readings between such conductors, or even a reading between the left conductor 712 and the middle conductor 712. However, the sensor 700 does not detect a health condition.

As shown by reference number 760 in FIG. 7C, wetness may alternately be present in a condition that activates at least one health condition sensor 716 based on a health condition. For example, a wetness including a predetermined material or substance, such as nitrite for the detection of UTI, may be is determined by one health condition sensor 716 (e.g., that associated with the middle conductor 712) and a wetness including a bacteria may be determined by another health condition sensor 716 (e.g., that associated with the left conductor 712. As wetness is present, the sensor 600 changes state, but also as indicated by 670, a health condition is determined.

As shown by reference number 770 in FIG. 7C, the sensor 700 determines a change in at least one of the health condition sensors 716 indicating that a health condition is present. Change either health condition sensor 616 may change the character of conduction through the respective conductor 712 (e.g., conduction through a carbon nanotube, other carbon allotrope, chemiresistor 716, etc. may increase or decrease relative to conduction through wetness alone, detectable as a change in conductance, resistance, or impedance). Changes in the readings of both health condition sensors in the foregoing example may enhance the likelihood that the determination of the health condition is correct. Alternately, for further example, one of the health condition sensors may be selected for determining a different urinary health condition, such as dehydration, such that the sensor is multifunctional for multiple urinary or other health conditions. It will be appreciated that greater numbers of conductors 712 and greater numbers of health condition sensors 716 may be included so as to provide for enhanced likelihood of correct determinations, broader scope of determinable health conditions, or combinations thereof.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the present disclosure. For example, it is to be understood that the present disclosure contemplates that, to the extent possible, one or more features of any embodiment or aspect can be combined with one or more features of any other embodiment or aspect. In fact, many of these features can be combined in ways not specifically recited and/or disclosed in the specification.

What is claimed is:

1. A sensor for detecting the presence of a health condition in a patient, the sensor comprising:
an absorbent garment or pad;
a transmitter including one or more processors; and
at least one conductor comprising one or more segments that are separated by absorbent material of the absorbent garment or pad, the at least one conductor including one or more health condition sensors, wherein the at least one conductor is configured to detect wetness in the absorbent garment or pad, and wherein the one or more health condition sensors are configured to detect a urinary health condition in the wetness.

2. The sensor of claim 1, wherein the one or more health condition sensors comprise a carbon allotrope modified with a diazonium compound.

3. The sensor of claim 2, wherein the carbon allotrope is a carbon nanotube.

4. The sensor of claim 3, wherein the carbon nanotube is a single walled carbon nanotube.

5. The sensor of claim 2, wherein the carbon allotrope is graphite.

6. The sensor of claim 1, wherein the at least one conductor comprises a carbon allotrope.

7. The sensor of claim 6, wherein the carbon allotrope is graphite.

8. The sensor of claim 1, wherein the health condition sensor is configured to detect the urinary health condition based on an electrical resistance or impedance in the health condition sensor changing when the at least one conductor and the health condition sensor are exposed to human urine including a bacteria.

9. The sensor of claim 1, wherein the health condition sensor is configured to detect the urinary health condition based on an electrical resistance or impedance in the health condition sensor changing when the at least one conductor and the health condition sensor are exposed to human urine including an elevated level of nitrite.

10. The sensor of claim 1, wherein the health condition sensor is configured to detect the urinary health condition based on an electrical resistance or impedance in the health condition sensor changing when the at least one conductor and the health condition sensor are exposed to human urine including a depressed pH.

11. The sensor of claim 1, wherein power is supplied to the at least one conductor to detect wetness in the absorbent garment or pad by:
a) forming a circuit based on wetness bridging between a first segment of the one or more segments and a second segment of the one or more segments, when urine is absorbed in the absorbent material, wherein the first segment and second segment are connected; or
b) forming a circuit based on wetness bridging between a first segment of the one or more segments and a second segment of the one or more segments, when urine is absorbed in the absorbent material, wherein the first segment and second segments are individual segments connected in parallel to the transmitter, and wherein absent wetness no circuit exists between the first and second segments.

12. The sensor of claim 1, wherein the one or more health condition sensors are configured to change in conductivity when activated with a bacteria, wherein a first state is associated with a first resistance or impedance and a second state is associated with a second resistance or impedance.

13. The sensor of claim 12, wherein the conductor comprises a continuous circuit including an initial impedance, wherein wetness including a bacteria on the at least one first conductor changes the impedance to a first state and then a second state, and wherein the first and second impedance are different from the initial impedance.

14. A health condition detection method, comprising: providing a sensor, in a computing system including one or more processors, the sensor including a conductor in an absorbent garment or pad, and the conductor including one or more health condition sensors responsive to a bacteria, material, or substance present in urine and associated with one or more urinary health conditions, wherein power is supplied to the conductor to form a circuit; detecting, by the computing system, wetness in the absorbent garment or pad; detecting, by the computing system, the bacteria, material, or substance; and determining, by the computing system, the urinary health condition based on the bacteria, material, or substance.

15. The health condition detection method of claim 14, wherein the one or more health condition sensors comprise a carbon allotrope modified with a diazonium compound.

16. The health condition detection method of claim 15, wherein detecting the bacteria further comprises: determining a sensor reading in the one or more health condition sensors based on an activated state of the carbon allotrope.

17. The health condition detection method of claim 16, further comprising: determining that the carbon allotrope is in the activated state by comparing a conductivity of the sensor with a previous reading, a stored reading, or a threshold reading of the sensor.
